(19) United States
(12) Patent Application Publication
(10) Pub. No.: US 2012/0213856 A1
(43) Pub. Date: Aug. 23, 2012

(54) Manufacturing Method of Microcapsule

(76) Inventors: Po-Lun Wang, Taipei City (TW); Shorong-Shii Liou, Kaohsiung City (TW); I-Min Liu, Kaohsiung city (TW); Chia-Ju Chang, Kaohsiung City (TW)

(21) Appl. No.: 13/032,701

(22) Filed: Feb. 23, 2011

(51) Int. Cl. A61K 9/50; A61P 43/00; A61K 36/232

(52) U.S. Cl. 424/490; 424/725; 424/493

(57) ABSTRACT

A manufacture method of microcapsule, comprises a step of extraction, by extracting active substance from a sample of Chinese angelica and obtaining an extract; a step of encapsulation, by encapsulating the extract with a covering, wherein the extract and the covering are mixed and co-dropped in a solidified solution for solidifying the covering, and the extract is encapsulated in the solidified covering to obtain a microcapsule; and a step of drying, by drying and finalizing the microcapsule.

### BACKGROUND OF THE INVENTION

1. Field of the Invention

The present invention relates to a manufacture method of microcapsule and, more particularly, to a manufacture method of microcapsule, which can preserve active substance of Chinese angelica.

2. Description of the Related Art

Angelica sinensis Diels, also known as “Chinese angelica”, “dong quai”, “climbing fig”, “ligusticum”, or “female ginseng”, is a perennial herb of Apiaceae family and is widely used in Chinese traditional medicine to treat for anemia, gynecological ailments, and flatulence. Generally, the Chinese angelica is used in the forms of a decoction or powder of traditional medicine. Hence, a time-consuming and complicated process of refining or decocting is needed to extract active substance from the Chinese angelica.

However, due to the less stability of the active substance in the Chinese angelica, ferulic acid and ligustilide in particular, those active substances will easily be degenerated under various temperature and humidity during the complicated process of refining or decocting of traditional medicine. Accordingly, the active substance in the Chinese angelica will dramatically diminish, so that the therapeutic effects of the Chinese angelica will also be reduced.

Although extraction can directly obtain a great amount of the active substance from the Chinese angelica without going through the refining or decocting process of traditional medicine, the active substance, ligustilide in particular, in the extracted solution is unstable and easy to degenerate during preservation. Furthermore, the active substance in the extracted solution are susceptible to hydrochloric acid in human gastric juice, so that only a small amount of the active substance can be taken from intestine. As a result, the extracted solution of the Chinese angelica is still poor in use. The extraction is also an improper method to obtain and preserve active substance from the Chinese angelica.

Therefore, there is a necessity of improving the disadvantage of conventional art and providing a new approach for successfully obtaining and stably preserving the active substance of the Chinese angelica.

### SUMMARY OF THE INVENTION

The primary objective of this invention is to provide a manufacture method of microcapsule, which can stably preserve active substance of Chinese angelica in microcapsule.

The secondary objective of this invention is to provide a manufacture method of microcapsule, which cause no damage to active substance of Chinese angelica and can successfully maintain the therapeutic effects of Chinese angelica.

The other objective of this invention is to provide a manufacture method of microcapsule so as to be convenient in process and frugal in energy use. A manufacture method of microcapsule, comprises a step of extraction, by extracting active substance from a sample of Chinese angelica and obtaining an extract; a step of encapsulation, by encapsulating the extract with a covering, wherein the extract and the covering are mixed and co-dropped in a solidified solution for solidifying the covering, and the extract is encapsulated in the solidified covering to obtain a microcapsule; and a step of drying, by drying and finalizing the microcapsule.

Further scope of the applicability of the present invention will become apparent from the detailed description given hereinafter. However, it should be understood that the detailed description and specific examples, while indicating preferable embodiments of the invention, are given by way of illustration only, since various others will become apparent from this detailed description to those skilled in the art.

### BRIEF DESCRIPTION OF THE DRAWINGS

The present invention will become more fully understood from the detailed description given herein below and the accompanying drawings which are given by way of illustration only, and thus are not limitative of the present invention, and wherein:

FIG. 1 is a diagram illustrating procedures of a manufacturing method of microcapsule;

FIG. 2 is a cross sectional view illustrating a soft gelatin encapsulation machine of a preferable embodiment in the present invention.

All figures are drawn for ease of explaining the basic teachings of the present invention only; the extensions of the figures with respect to number, position, relationship, and dimensions of the parts to form the preferred embodiment will be explained or will be within the skill of the art after the following teachings of the present invention have been read and understood. Further, the exact dimensions and dimensional proportions conforming to specific force, weight, strength, and similar requirements will likewise be within the skill of the art after the following teachings of the present invention have been read and understood.

Where used in the various figures of the drawings, the same numerals designate the same or similar parts. Furthermore, when the terms “first”, “second”, “inner”, “end”, “portion”, “section”, “top”, “bottom”, “axial”, “radial”, “spacing”, and similar terms are used herein, it should be understood that these terms refer only to the structure shown in the drawings as it would appear to a person viewing the drawings, and are utilized only to facilitate describing the invention.

### DETAILED DESCRIPTION OF THE INVENTION

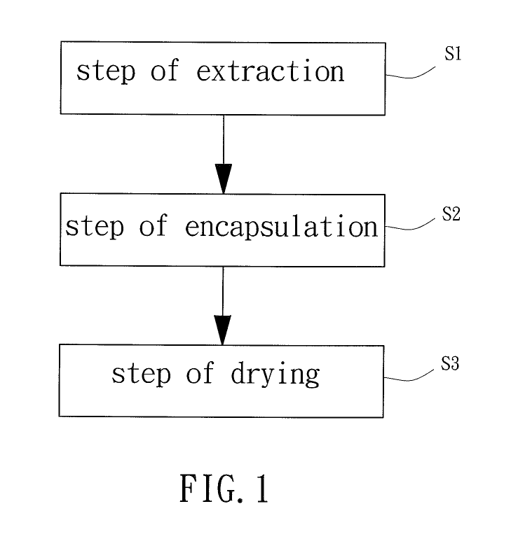

The present invention is a manufacturing method of microcapsule, which can preserve active substance of Chinese angelica, ferulic acid and ligustilide in particular in a microcapsule. Referring to FIG. 1, the manufacturing method of microcapsule of the present invention comprises a step of “traction S1”, a step of “encapsulation S2” and a step of “drying S3”.

In the step of “extraction S1”, the active substance of the Chinese angelica is extracted from a sample of Chinese angelica to obtain an extract.

The sample can be one of Angelica acutiloba Kitagawa, Angelica actiloba Kitagawa var. sugiyamae Hikino, Angelica acutiloba Max and Angelica acutiloba Oliv. In the present embodiment, roots of Angelica acutiloba Kitagawa are prepared and extracted in the step of “extraction S1”.

Precisely, the root of Angelica acutiloba Kitagawa is ground into powder, followed by extracting the angelica powder via supercritical carbon dioxide extraction. In the present embodiment, a 1000 bar extraction pilotplant system is used and processed at a condition of 30 kg/hr to 35 kg/hr of flow rate of supercritical carbon dioxide, 200 bar to 652 bar of pressure and 40° C. to 65° C. of temperature for one to two hours, in order to obtain the extract. The extract is in the form of wax and contains several active substances, ferulic acid and ligustilide in particular.

Preferably, the extract of the present embodiment can be 10 to 100 times diluted by an edible oil, such as olive oil, camellia oil, sesame oil, flax oil, mustard oil, soybean oil peanut oil, sunflower oil, corn germ oil, seabuckthom oil, palm oil and fish oil. With such dilution, the active substance can be dissolved in the edible oil. The liquid phase of the extract will be easy and convenient to process in the follow-up steps. Moreover, with the above arrangement, the active substance of the Chinese angelica will be stable and uneasily to degenerate, and also the fragrance of the Chinese angelica can be perfectly maintained.

In the preferably embodiment, the extract is mixed with the edible oil in a weight ratio of 48:1000, and kept at 40° C. to 50° C. for facilitating the solubility of the active substance in the edible oil. In this way, the active substance in the extract dissolved in the edible oil will become less easy to degenerate, but easy to manufacture and process.

In the step of “encapsulation S2”, the extract is encapsulated in a covering, by co-dropping the extract and the covering into a solidified solution for solidifying the covering to cover the extract and to obtain a microcapsule. Precisely, the step of “encapsulation S2” is processed at 40° C. to 50° C. The covering is an anion solution, and the solidified solution is a duad cation solution, such as Ca²⁺, Sr²⁺, Ba²⁺, Ni²⁺, Cu²⁺, and Zn ²⁺. In the present embodiment, the covering is sodium alginate with a weight percent of 1.2% to 2.0%, and the solidified solution is calcium dichloride with a weight percent of 1.5% to 3.0%. The extract and the covering are dropped into the solidified solution at the same time. With such arrangement, the covering will solidify and stably cover the extract due to a fast crosslink-gelating relation between the anion solution (the covering) and the cation solution (the extract), and finally the microcapsule is obtained.

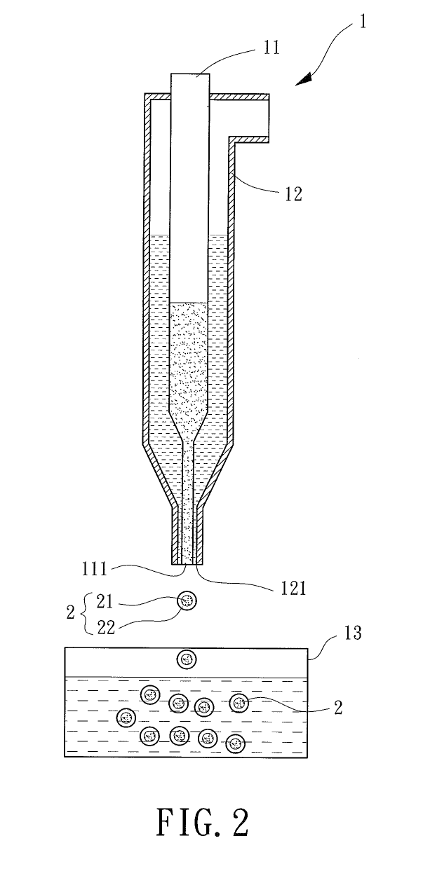

Particularly, the microcapsule of the present invention is manufactured by a piercing-solidifying method in the step of “encapsulation S2” of the present embodiment. As an example, a softgel encapsulation machine is prepared and used for processing the step of “encapsulation S2”. Referring to FIG. 2, a soft gelatin encapsulation machine of the preferable embodiment is shown. The soft gelatin encapsulation machine 1 comprises a first dropper 11 with a first entrance 111, and a second dropper 12 with a second entrance 121. The first dropper 11 has smaller diameter than the second dropper 12, so that the first dropper 11 can be received in the second dropper 12, with the first entrance 111 in alignment with the second entrance 121. The soft gelatin encapsulation machine 1 further comprises a collector 13 containing the solidified solution. In practice use, the covering will flow into the collector 13 via the first dropper 11, and the extract will flow into the collector 13 at the same time via the second dropper 12. Accordingly, the covering will interact with the solidified solution and solidify to encapsulate the extract in the collector 13, and then a microcapsule 2 will be obtained. As it is shown in FIG. 2, the microcapsule 2 has a core 21 and a housing 22, wherein the core 21 consists of the extract and housing 22 consists of the covering.

Precisely, the first entrance 111 and the second entrance 121 of the soft gelatin encapsulation machine 1 is various from 0.5 to 1.0 mm in diameter. Furthermore, the flow rate of the soft gelatin encapsulation machine 1 is adjustable according to the objective diameter of the microcapsule 2, which is around 1.5 mm to 5.0 mm. In the present embodiment, the flow rate of the soft gelatin encapsulation machine 1 is set up at 120 drops per minute, in order to obtain the microcapsule 2 of 4.0 mm. Additionally, the soft gelatin encapsulation machine 1 can further comprise a adjusting module for adjusting the temperature at about 40° C. to 50° C. In this way, not only the liquidity of the covering can be maintained, but also the degradation of the active substance in the extract will not be seriously improved.

In the step of “drying S3”, the obtained microcapsule of the present invention is drying and finalizing in structure. In the present invention, the microcapsule 2 obtained from the step of “encapsulation S2” is wet and needs to be dried at 20° C. to 50° C. For example, the microcapsule of the present invention can be either kept at room temperature for drying, or dried by dehumidifying the room humidity to less than 20%. With such process, the water of the microcapsule will be removed so that the microcapsule can get dry and stable in structure. Accordingly, the microcapsule for stably preserve active substance of the present invention is obtained, and which is easy to be absorbed by intestine.

Through the present invention, the manufacture method of microcapsule is provided, and which can successfully encapsulate the active substance of the Chinese angelica in the microcapsule for stable preservation and maintenance of therapeutic effects. In the step of “extraction”, the extract containing several active substances of the Chinese angelica, ferulic acid and ligustilide in particular, is extracted from the roots of the Chinese angelica. The extract is dissolved in the edible oil so as to be more stable and uneasily to oxidize and degrade. In the step of “encapsulation S2”, the extract of the Chinese angelica is encapsulated into the microcapsule for stable preservation and preventing from being degrade or lost of fragrance during processes. In the step of “drying S3”, the solidification of the covering will become far more stable and firm so that the structure of the microcapsule can be finalized for perfectly preserving the active substance of the Chinese angelica, as well as persistently maintaining the therapeutic effects of the Chinese angelica.

With such arrangement, the extract of the Chinese angelica not only can be well preserved in the microcapsule, but also can be smoothly delivered into human body with the microcapsule, avoiding being degraded by hydrochloric acid in gastric juice. Accordingly, a great amount of the active substance can be absorbed in intestine so that better therapeutic effects of the Chinese angelica can be achieved. Therefore, it is suggested that the manufacture method of the present invention has multiple advantages of easy to process and frugal in energy use. Also, with the manufacture method of the present invention, active substance of the Chinese angelica can be successfully obtained and stably preserved in the microcapsule.

Thus, since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

What is claimed is:

1. A manufacture method of microcapsule, comprising:
a step of extraction, by extracting active substance from a sample of Chinese angelica and obtaining an extract;
a step of encapsulation, by encapsulating the extract with a covering, wherein the extract and the covering are mixed and co-dropped into a solidified solution for solidifying the covering, and the extract is encapsulated in the solidified covering to obtain a microcapsule; and
a step of drying, by drying and finalizing the microcapsule.

2. The manufacture method of microcapsule as defined in claim 1, wherein the microcapsule has a core and a housing, with the extract being the core and with the covering being the housing.

3. The manufacture method of microcapsule as defined in claim 2, wherein the extract has ferulic acid and ligustilide.

4. The manufacture method of microcapsule as defined in claim 2, wherein the covering is sodium alginate.

5. The manufacture method of microcapsule as defined in claim 2, wherein the solidifying solution is calcium dichloride.

6. The manufacture method of microcapsule as defined in claim 1, wherein the step of extraction is processed at 40° C. to 65° C.

7. The manufacture method of microcapsule as defined in claim 1, wherein the step of encapsulation is processed at 40° C. to 50° C.

8. The manufacture method of microcapsule as defined in claim 1, wherein the step of drying is processed at 20° C. to 50° C.

9. The manufacture method of microcapsule as defined in claim 8, wherein a process of dehumidifying is further performed before the step of drying, by adjusting the humidity to less than 20% for removing water on the microcapsule.

10. The manufacture method of microcapsule as defined in claim 1, wherein a process of dilution is further performed after the step of extraction and before the step of encapsulation, by diluting the extract in 10 to 100 times weight of an edible oil.

11. The manufacture method of microcapsule as defined in claim 10, with the edible oil being one of olive oil, camellia oil, sesame oil, flax oil, mustard oil, soybean oil peanut oil, sunflower oil, corn germ oil, seabuckthorn oil, palm oil and fish oil.
